(19) United States
(12) Patent Application Publication
(10) Pub. No.: US 2006/0099154 A1
(43) Pub. Date: May 11, 2006

(54) Teeth whitening method, products, system and method of conducting business thereby

(76) Inventor: Joey Kahwaty, Renton, WA (US)

Correspondence Address: John P. Luther, Esq.;Newman & Newman, LLP, Suite 610, 505 Fifth Avenue South, Seattle, WA, 98104, US

(21) Appl. No.: 10/985,387

(22) Filed: Nov. 9, 2004

(51) Int. Cl. A61K 8/22

(52) U.S. Cl. 424/53

(57) ABSTRACT

The present invention provides an improved dental whitening method and composition, and method of conducting business thereby, having reduced teeth and oral tissue sensitivity, which comprises the application of a whitening composition, which is either preceded or followed by the application of a teeth desensitizing composition, and optionally the prior, simultaneous or subsequent application of additional dental active compounds, adjuvants or excipients. The whitening and desensitizing compositions are discreetly and separately applied, and in preferred embodiments are applied from custom made trays fabricated from dental impressions taken or made by the user.

### FIELD OF THE INVENTION

The present invention relates to teeth whitening methods, a system for implementing such methods, products for use in such methods, and methods for conducting business inclusive of such methods, products, and system, and particularly to teeth whitening and desensitizing products and methods of use.

### BACKGROUND OF THE INVENTION

Copyright Notice 2004 Joseph Kahwaty. A portion of the disclosure of this patent application/patent document contains material which is subject to copyright protection. The copyright owner has no objection to the facsimile reproduction by anyone of the patent document or the patent disclosure as it appears in the U.S. Patent and Trademark Office file or records.

A variety of substances that a person may ingest and which come in contact with teeth over a period of time, such as coffee, tea, certain foods and tobacco, may gradually and permanently stain or discolor the teeth. Eventually such staining and/or discoloration may proceed to a degree that subjectively imparts feelings of unattractiveness to the person. Such a condition may even affect the way a person is treated or perceived by others. As such staining becomes more or less permanent, mere brushing will not remove it. At this point usually only chemical methods, such as bleaching and the like, will remove the oftentimes decades worth of staining and/or discoloration. Thus, teeth whitening and cosmetic dental care in general has become a major industry in recent years with the addition of many players and an interesting array of new products and procedures, such as teeth whitening procedures, whitening gels and pastes, whitening trays and the like, all of which are available from both dental offices and retail sources such as the Internet.

For example, as disclosed in U.S. Patent Application publication No. 20040062723, a peroxide-based dual component dentifrice composition said to provide enhanced whitening efficacy is provided. This composition is comprised of a separately maintained first aqueous dentifrice component containing a peroxide compound and an abrasive compound system compatible with the peroxide and a second aqueous dentifrice component containing a desensitizing compound and an abrasive that is incompatible with the peroxide compound. When the components are mixed and combined for the first time and simultaneously exposed to the surface of teeth, enhanced whitening is said to be obtained along with minimal tooth sensitivity and gum imitation. The peroxide component is hydrogen peroxide in relatively low concentrations with the abrasive being calcium pyrophosphate. The desensitizing component can be potassium salt, and is used with an abrasive such as silica or alumina. As further described, the individual components being incompatible with each other are only combined and admixed at the moment they are simultaneously applied to teeth by a patient, and which is said to provide an unexpectedly rapid teeth whitening.

A teeth whitening kit is disclosed in U.S. Patent Application Publication No. 20020146666. In this publication the kit is described as including a package which contains a tooth whitening composition with a peroxide-based compound and a set of instructions for use of the package's components to minimize soft tissue irritation. Among other things, there are instructions not to brush the teeth and/or soft tissue prior to applying a teeth whitening composition, and preferably to avoid brushing for up to sixty minutes before undertaking any teeth whitening to avoid teeth and tissue sensitivity. The kit is also described as containing toothpaste and a toothbrush for brushing after the application of a tooth whitening composition. As also mentioned in this method, the avoidance of brushing is to avoid the formation of chemical radicals when a prior used toothpaste pH environment of a basic nature is in contact with a teeth whitening composition applied to the teeth. These radicals are thought to be responsible for increased gum irritation during the whitening process. Also disclosed is the use of desensitizing agents contained in the teeth whitening composition, which may be potassium nitrate, citric acid, citric acid salts, strontium chloride, and various combinations thereof. A variety of methods to apply the whitening composition can include a tray, a stint, a brush or pen application, doe's foot applicator, a person's finger, a cotton swab or a dental stick and the like. Additionally, this composition may also contain a pH adjusting agent, such as sodium bicarbonate, sodium phosphate, sodium hydroxide or potassium hydroxide, etc., in an effort to ensure that the teeth whitening composition is safe for oral tissues and/or may function as a buffer to effect adjustment of the pH of the teeth whitening composition as applied.

More teeth whitening compositions and methods are described in International Application No. PCT/US99/26355 0028953, along with methods and products for application, such as trays, syringes and/or squeezable tubes. As disclosed, the compositions include sticky and viscous teeth whitening agents, such as hydrogen peroxide or carbamide-based hydrogen peroxide, and may also include teeth sensitivity reducing agents, such as potassium nitrate, for its simultaneous application with the teeth whitening peroxides to provide both opacification and desensitization of teeth and mouth tissues. These compositions are also said to be substantially free of abrasives, as such are thought to interfere with teeth whitening active ingredients or their application, in contrast to above-mentioned compositions. Additionally, the compositions are preferably used with a thin-walled, flexible and lightweight dental tray to hold the composition in a position adjacent to dental surfaces.

Yet additional compositions and methods for whitening and desensitizing teeth are disclosed in International Application No. PCT/US01/02945 0154610. Here, as in some other compositions, the whitening compositions and methods include both a dental bleaching agent and potassium nitrate for enhanced whitening and reduced teeth sensitivity. The simultaneous application of potassium nitrate along with the teeth whitening agent is thought to enhance the whiteness of teeth beyond that of the whitening efficacy of the bleaching agent alone, in addition to eliminating or reducing teeth sensitivity. As further discussed, during the course of prolonged teeth treatment, lower quantities of potassium nitrate are thought to be more effective for both whitening and desensitizing simultaneously. These compositions may be applied directly to a person's teeth, or may be loaded into a thin-walled tray and placed over a person's teeth. When used with a tray, the compositions include a tackifying agent, such as carboxypolymethylene, which is thought to assist the composition in retaining the dental tray over a person's teeth; and which is also thought to be superior to the mere mechanical interlocking of a tray over a person's teeth. The compositions may also include anticarlogenic and anti-microbial agents.

While not specifically related to teeth whitening compositions, but instructive of conventional art nevertheless, U.S. Pat. No. 5,505,933 discloses a desensitizing anti-tarter dentifrice, which is delivered or applied as toothpaste. The anti-tarter agent may be a polyphosphonate compound, preferably used in conjunction with a synthetic anionic polymeric polycarboxylate or equivalent compounds and fluoride. The composition may also include as a proportion a desensitizing potassium salt which my pass through exposed dentin tubules to tooth nerves or neurons, such as potassium nitrate, potassium citrate or potassium oxalate.

For use with such compositions and methods described above, an improved dental impression tray is disclosed in U.S. Pat. No. 6,749,428. This product is said to provide an improved retentive force for dental impression material that it contains by incorporating a plurality of projections and panels on its tray walls. Upon taking a dental impression, the dental impression material flows into the grooves defined by a plurality of projections, panels, and tray walls to form retentive components which prevent the impression material in the tray from being distorted or lifted out of the tray when it is disengaged from the oral anatomy of the patient. The end result is a well formed dental impression which can be effectively employed as a bleaching tray or other dental product.

While many no doubt effective teeth whitening compositions and those with desensitizing components are available, as shown by the several examples above, there is still a great need and much room for improved teeth whitening compositions and methods which can be used without causing teeth and mouth tissues to be overly sensitized.

There also exists an important need for such improved teeth whitening/bleaching compositions and methods which can be conveniently sold and distributed by both dental offices and/or retail outlets alike and conveniently used and self-applied by a person for rapid and effective teeth whitening without the tendency of bleaching agents to cause teeth and mouth tissues to become overly sensitized.

There further exists an important need for the maintenance of a system or business model which incorporates such improved and superior methods for conducting teeth whitening and desensitizing, and a dental products business in general, which may economically serve many patients simultaneously.

### SUMMARY OF THE INVENTION

In accordance with those needs and desires as mentioned above, the present invention provides an improved dental whitening method and composition having reduced teeth/oral tissue sensitivity which comprises the application of a teeth whitening composition, preceded and/or followed by the application of a teeth desensitizing composition, and optionally the prior, simultaneous or subsequent application of additional dental active compositions or compounds, adjuvants and excipients, such as fluoride, anti-bacterial, anti-fungal and/or remineralizing agents and the like. The whitening and desensitizing compositions are discretely and separately applied in order, and in preferred embodiments in their own custom trays made from dental impressions manufactured and obtained from the customer h/herself, in contrast to tray impressions produced in a dental office by a dental professional.

The present invention, including its products, methods of application, total system and methods of conducting a business thereby will be more fully understood with reference to the following Detailed Described of Preferred Embodiment's.

### DETAILED DESCRIPTION OF PREFERRED EMBODIMENTS

All patent references, published patent applications and literature references referred to or cited herein are expressly incorporated by reference herein to the same extent as if each were specifically and individually indicated to be incorporated by reference. Any inconsistency between these publications and the present disclosure is intended to and shall be resolved in favor of the present disclosure.

### I. Teeth Whitening

In its broadest sense, the present invention encompasses a teeth whitening, which is applied to a persons' teeth as desired, and which is proceeded and/or followed by the separate and discrete application of a teeth desensitizing and/or oral tissue desensitizing composition, with each composition applied by a person, or by another to the person, by way of dental product application means, or in a preferred embodiment by custom made dental impression trays manufactured from impressions produced and provided by the user/patient.

Teeth whitening compositions useful in this invention are generally known in the marketplace, and most, if not all, are suitable for use as whitening or bleaching compositions herein. Generally, dental or teeth whitening compositions are peroxide-based, and are preferably used in gel form with the present invention. However, any conventional teeth whitening composition is suitable for use herein notwithstanding whether one particular composition is more effective in whitening or bleaching teeth than another in a particular environment, such as within a given pH range. Whether in gel form or some other physical form, such teeth whitening compositions may be applied by any conventional or non-conventional method, such as with the custom made application trays discussed more fully below, or by a dental strip means containing a whitening composition to be held in contact with teeth for a period of time, or by an application stick or cotton ball and the like. The dental whitening compositions may be pre-applied to an applicator means, or may be applied to the applicator means by a user, for example, by way of a syringe or a squeezable tube with the means and method of teeth whitening composition application not being critical to the practice of this invention.

The particular teeth whitening composition employed is also not critical to the practice of this invention, with the only caveat being that some compositions are, or course, more preferred than others, which may also depend upon several considerations including the conditions under which a composition is employed, such as pH, temperature and time factors, and how the composition is applied and its frequency of application.

Some examples of several teeth whitening compositions suitable for use herein may be found in U.S. Patent Application Publication No. 20022014666, and U.S. Pat. Nos. 6,136,297; 5,376,006; 5,985,249; 5,922,307; Re 34,196 and International Application Publication No. WO. 99/62472. While virtually any peroxide-based compound suitable for dental use is contemplated for use herein, some specific examples of peroxide-based substances capable of forming oxygen radicals which may be used include hydrogen peroxide, organic peroxides, such as carbamide peroxide, glycerol peroxide, benzoyl peroxide or metal ion-containing peroxides, such as calcium carbonate peroxide or sodium carbonate peroxide, and various mixtures thereof. Carbamide peroxide is considered the most preferred for use in this invention, particularly in preferred gel forms of the teeth whitening composition.

Additional examples of other teeth whitening compositions which may be employed include compositions which can produce oxygen radicals, for example, hydrogen peroxide when admixed with an aqueous substance, such as, for instance, percarbonates such as sodium percarbonate and mixtures thereof with other peroxide-containing or liberating compounds.

While it is contemplated in this invention that a peroxide-based, or peroxide producing or generating component or compound, may be present in a teeth whitening composition in any amount effective for teeth whitening, some considerations for preferred amounts may be (1) the degree of teeth whitening sought to be obtained through use of the inventive compositions and methods, and other dependant considerations, such as, without limitation, (2) the length of time a teeth whitening agent is contemplated for contact with a users' teeth, and (3) the frequency of application of a teeth whitening composition. As may be envisioned, any sort of teeth whitening schedule or regimen may be devised and/or tailored, for example, to the personal habits and lifestyle of the user(s) and for h/her particular response to a treatment regimen. Treatment adjustments, depending upon the whitening optimization sought in a certain regimen with a particular teeth whitening composition, are certainly contemplated within the practice of the invention. In general, however it is preferred that a concentration of hydrogen peroxide equivalent be present in a teeth whitening composition from between about 0.10% by weight to about 50% by weight, and more preferably from about 10 to about 35% by weight, with a particularly preferred carbamide peroxide concentration range comprising up to about 22 to about 25% by weight of the teeth whitening composition.

Other ingredients of the teeth whitening composition may be any conventional or non-conventional compound or material of any sort which is responsible for any sort of a display of efficacy for any end result contemplated, such as additional dental active products, adjuvants and excipients, and vehicles or solvents for such compounds. Some non-limiting examples include water, gelling agents, pH adjusting agents (sometimes referred to as buffers, or buffering agents), stabilizers, activators, promoters, catalysts, accelerating agents, flavoring agents, sweetening agents, such as saccharin, coloring agents such as FD&C approved dyes and chelating compounds, such as ethylene diamine-tetraacetic acid (EDTA), and any and all admixtures of such materials and substances.

Gelling agents are generally preferred for use with the teeth whitening compositions of this invention due, at least in part, to their ease of use and homogeneous coverage of surface areas sought to be treated. Such gelling agents may also be chosen to have a particular viscosity which allows them to adhere better to the surfaces to which they are applied. When choosing a gelling agent, depending upon the make-up and nature of the teeth whitening composition contemplated (and the use of other additives, adjuvants and excipients) care should be exercised that a non-reactive gelling agent is selected. While some consideration may be entailed in this selection, by no means is an undue amount of experimentation required given the well known nature and use of conventional gelling agents, especially in the dental care products field. Here, some non-limiting examples may include polymeric materials which may be either water soluble or water insoluble as well as those of limited solubility, such as polyvinylpyrrolidone, carboxymethylcellulose, carboxypropylcellulose as well as derivatives thereof, such as diehtylaminoethyl or triethylaminoethyl cellulosic compounds carboxypolymethylene, hydroxymethylcellulose, hydroxyethylcellulose, methylcellulose, methyl hydroxyethylcellulose, methyl hydroxypropylcellulose, ethyl hydroxyethylcellulose all of which can be modified by addition to one another, and natural and synthetic gums, such as alginates, carrageenan, gum karaya, xanthan gum, guar gum, gum arabic, gum tragucanth, carboxyvinyl polymers, poloxamer, and pectins from fruits. The amount of gelling agent present or used to manufacture the composition may be from about 0.001% by weight to in excess of about 50% by weight of the teeth whitening composition, depending upon the compositions' manufacture, uses contemplated and the physical nature of the other constituents present, and is usually preferred for use in amounts from between about 00.1% by weight to about 20% by weight. A preferred gelling agent for use in the composition is carboxymethylcellulose which is present in a range of from about 0.10% by weight to about 15% by weight of the composition.

One or more thickening agents may also be present, which may also act as gelling agents. Some non-limiting examples include starches, silica, such as amorphous silica, natural and synthetic gums, such as carrageenan, guar gum and the like, hydroxyethylpropylcellulose, hydroxybutylmethylcellulose, hydroxypropylmethylcellulose, and sodium salts of carboxymethyl cellulose and various mixtures thereof. The amount of thickener(s) employed may be present in any amount required to achieve a desired viscosity with or without the presence of a gelling agent, and may range from about 0.001% by weight to about 20% by weight of the composition.

Some non-limiting examples of stabilizing agents include parabens, such as methyl and propyl paraben and/or sodium salts thereof. Some non-limiting examples of preservatives may include benzoic acid and perhaps salicylic acid, butylated hydroxylated-anisole (BHA) butylated hydroxytoluene (BHT), phosphates and tin salts. These agents are preferred for use in amounts ranging from about 0.001% by weight to about 20% by weight.

Buffering or pH adjusting material suitable for use in this invention may be any material effective to adjust the pH of the teeth whitening composition as desired, such as to ensure that it is safe for oral tissues. Some non-limiting examples of pH materials with which pH might be adjusted include sodium hydroxide, ammonium hydroxide, potassium hydroxide, sodium stannate, triethanolamine, citric acid, hydrochloric acid, sodium citrate, salts of carbonic acid such as sodium bicarbonate, salts of phosphate such as sodium and/or potassiium phosphate and sodium and or potassium dihydrogen phosphate, salts of tartaric acid as well as boric acid, imidazole, diethanolamine, glycine hydrocholoride, salts of hydrogen phthalate such as potassium hydrogenphthalate, salts of cacodylic acid and any combinations thereof. The amounts present in the teeth whitening composition are whatever is necessary to achieve and/or maintain a desired pH of the composition depending upon the other constituents which may be present, and the environment to which the whitening composition may be subjected.

Abrasive components of the teeth whitening composition are preferably compounds that are compatible with the peroxide-based compounds, such as described, for example, in U.S. Pat. No. 5,171,564. Some non-limiting examples include peroxide compatible calcium phosphate abrasives, such as dicalcium phosphate dihydrate, anhydrous dicalcium phosphate and calcium pyrophosphate. The abrasives may be present in the composition in concentrations from about 0.01% by weight to in excess of about 60% by weight, and preferably from about 0.10% to about 30% by weight. Conventional thought is that the presence of abrasive compounds is effective to increase the density of a peroxide-based component to better concentrate the available peroxide for increased whitening efficacy. Some examples of other suitable abrasives include hydroxyapatite, sodium metaphosphate, potassium metaphosphate, calcium carbonate, sodium bicarbonate, alumina trihydrate, alumina silicate and calcinated alumina, hydrated silica compounds (I will be more specific on this shortly. Also should we say something about particle size here, as particle size is an issue in the rheological properties of the gel) While on the rheological properties should we make claims as to preferred rheological properties.

Another class of compounds known to increase teeth whitening efficacy are chelating agents, which are thought to improve or enable peroxide storage stability, oftentimes referred to as shelf life. Some non-limiting examples of these compounds suitable for use herein may include, for instance, sodium acid pyrophosphate, disodium calcium ethylene diamine tetracetic acid (Na₂Ca EDTA), phosphoric acid, citric acid, sodium citrate, potassium citrate, sodium phosphate, sodium pyrophosphate, potassium pyrophosphate, potassium stannate, magnesium dipotassium ethylene diamine tetracetate and disodium ethylene-diamine tetracetate. As known in the art, the presence of one or more chelating agents is thought to inactivate any metal impurities present which may tend to destabilize a peroxide-based compound. The chelating agent can be incorporated into the teeth whitening compound during manufacture along with other constituents, or it may be obtained in a commercially prepared admixture with a peroxide-based compound. In any event, such chelating agents are generally present in the teeth whitening composition in a chelating amount, or perhaps in a concentration ranging from about 0.01% by weight to about 30% by weight, or in any amount compatible with other teeth whitening composition ingredients and found effective to achieve an acceptable product shelf life for desired teeth whitening efficacy.

The use of surfactants is also contemplated herein to provide wetting properties when desired, to ensure that the whitening composition reaches the cracks, crevices and interstitial spaces of teeth for a far reaching and homogeneous whitening effect. These surfactants may be combined with foaming agents to further ensure or enhance the homogenous whitening effect of the whitening composition. Depending upon the teeth whitening composition constituents employed, and the properties thereof contemplated, any of the several known anionic, non-ionic, zwitterionic and cationic surfactants, and foaming agents may be employed, or any combination thereof. Some non-limiting examples of nonionic surfactants useful herein include alcohol ethoxylates and the alkylphenol ethoxylates, alkyl polyglycosides and sorbitan ester surfactants, ethoxylated sorbitan esters as well as nonethoxylated sorbitan esters. Examples of zwitterionic surfactants include ammonium carboxylates, ammonium sulfates and amine oxides.

One or more surfactants and/or foaming agents may be employed in the teeth whitening composition in virtually any amount found to achieve the desired properties of the composition, but are generally employed in relatively small amounts, such as from about 0.01% by weight to about 5% by weight.

Foaming agents doubling also as hybrid surfactants may also be used to enhance efficacy of the whitening compositions. An example of a suitable hybrid surfactant is sodium laureth sulfate. Other foaming agents might include higher alkyl sulfates, such as potassium or sodium lauryl sulfate, fatty acid monoglycerides of hydrogenated coconut oil fatty acids, alkylaryl sulfonates, such as sodium dodecyl benzene sulfonate and fatty sulfoacetates and fatty esters of 1,2 dihydroxy propane. Additional examples of surfactants include the sodium salts of oleic, palmitic, myristic and stearic acids.

Colorants, dyes, flavoring, and sweeteners may also be employed. Desirable colorants, such as pigments and dyes, are, of course, non-toxic, and may include food color additives certified for ingestion, or otherwise oral deployment, in accordance with the U.S. Food, Drug and Cosmetic Act. Some non-limiting examples include FD&C Red No. 3, FD&C Red No. 3 (sodium salt of tetraiodofluorescein), FD&C Yellow No. 5 (sodium salt of 4-p sulfophenylazo-1-p-sulfophentyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-[[4-(N-ethyl-p-sulffobenzylamino) (-3,5-cyclohexadienimine], FD&C Blue No. 1 (disodium salt of dibenzyldiethyldiaminotriphenylcarbinol trisulfonic acid of indigotin) and mixtures thereof in various proportions. The amounts of dye and pigments useful in the present whitening compositions may range widely from less than about 0.0001% by weight to in excess of about 30% by weight, but are generally preferred to be used in amounts up to about 1.0% by weight.

In a preferred embodiment of the teeth whitening compositions, peroxide sensitive dyes which react with and gradually lose their color upon contact with the peroxide ingredient may be employed to act as a signal to alert the user that the whitening treatment is completed and the mouth tray or other applicator and its contents may be removed from a user's teeth. Preferably, for example, components when initially combined are colored by the presence of the dye which color slowly fades over the treatment period. In such embodiments FD&C blue #1, FD&C blue #2, FD&C Green #3, Yellow #6, and FD&C red #40 are peroxide sensitive dyes which fade upon exposure to peroxide and are provided as useful examples. Of these, FD&C Green #3 and Yellow #6 are most preferred as color signals (blue or orange, respectively at alkaline pH), and gradually loose color upon exposure to a peroxide component over a desired treatment time, say, for example, from about 10 to about 30 minutes. Such peroxide reactive colorants may be combined with the teeth whitening composition just prior to its application to teeth such that it is not degraded prior to use.

Any suitable flavoring or sweetening material may also be incorporated in the whitening compositions. Examples of suitable flavoring constituents include flavoring oils, such as, for example, essential oils of spearmint, peppermint, wintergreen, sassafras, clove, sage, eucalyptus, marjoram, cinnamon, lemon, and orange and other citrus fruits and methyl salicylate. Other artificial or natural fruit flavors or extracts may also be employed. Nonlimiting suitable sweetening agents include sugars sucrose, dextrose, fructose, lactose, maltose, sorbitol, xylitol, sodium cyclamate, perillatine, aspartame and sodium saccharin. Preferably, flavor and sweetening agents may together comprise from about 0.01% to about 5% by weight or more of the compositions components, or whatever a user desires.

Still other active dental agents, adjuvants, and excipients may be included in the teeth whitening compositions of the invention to provide other types of dental and/or gum treatment, such as, for example, anti-cariogenic agents, of which xylitol present in an anti-cariogenic amount is an example. Other useful compounds include such as anti-demineralizing agents and remineralizing agents of which some non-limiting examples include fluoride salts, such as sodium fluoride, stannous fluoride, and sodium monofluorophosphate. Some gum disease fighting antimicrobial, antibacterial, anti-infective and/or antifungal agents, including agents generally known as antibiotics, which may be present include, for example, penicillins, chlorohexidine, tetracycline, cetyl pyridinium chloride, benzalkonium chloride, cetyl pyridinium bromide, methyl benzoate and benzoate and mixtures thereof. Any of such agents may be present in the teeth whitening composition in amounts which display efficacy for their intended purpose to any degree, or may be added in any efficacious amount to the whitening composition just prior to contact of the composition with teeth or after a teeth whitening session.

In still additional embodiments humectants, which may act as moisturizers by attracting moisture to themselves, may be added to the compositions. A preferred use is as a vehicle in the preparation of the compositions. For example, the humectants glycerin and polyethylene glycol admixed with water may be employed in preparing a vehicle for the peroxide-based component of the whitening compositions, to which other ingredients described above may be added. Preferably, however, care is taken to exclude water or moisture from the whitening compositions during and after preparation to avoid premature activation of the peroxide component. In another typical example preparation the peroxide whitening composition may be prepared a paste at ambient temperature by the use of anhydrous glycerin with a peroxide compound, such as the desired amount of carbamide peroxide, along with adjuvants and excipients, such as chelating agents, sweeteners and flavoring premixed with water until all solids are dissolved. Thickeners and/or stabilizers are separately processed and mixed until a uniform mixture is obtained, and then both admixtures are combined and stirred until a desired gel forms. Abrasives, such as calcium phosphate or various types of silica including hydrated silica compounds having a variety of particle sizes, ranging from submicron sizes to several microns, may then be added and the mixture stirred and/or processed until a uniform dispersion is obtained. While these methods are merely exemplary of some preferred embodiments of whitening composition preparation, the skilled artisan will undoubtedly find many other suitable methods without undue effort, depending upon the particular nature of the constituents employed.

### II. Teeth Desensitizing

Following application of the teeth whitening composition for a determined amount of time, such as for the degree of whitening efficacy desired or required, a users' sensitivity permitting, a separate and discrete application of a desensitizing composition to a user's teeth and gum areas is preferably undertaken. Optionally, such a desensitizing step may both proceed and/or follow a teeth whitening step. By proceeding teeth whitening, a desensitizing step may allow for a user to undergo a consistent and comfortable teeth whitening episode without interruption for better whitening effect.

As is known, the outer layer of teeth is comprised of enamel over an underlying and sensitive portion which includes any innervated portion of the teeth including the pulp of the tooth which is also supplied by blood and innervated. Teeth sensitivity may occur when the enamel is partially demineralized or made penetrable and/or gums recede, thereby exposing the dentin and nerves to external phenomena, such as temperature changes, changes in pH or the ionic environment of the teeth resulting from eating or drinking certain foods and/or beverages including sweet and/or sour foods which may also be acidic. The peroxide compounds used in the invention may also lead to sensitization of the teeth Due to free radical formation. Such exposure is thought to cause fluid movement as well as movement of ions into and through the tubules, with resulting stimulation of nerve fibers consequentially producing pain. It is also thought that dehydration of teeth, such as may occur with whitening/bleaching agents, may be a cause for teeth sensitivity and which is temporary and dissipates as teeth are rehydrated.

Several products and methods are known and available for treating teeth and gum sensitivity, and any of which alone or in combination are contemplated for use in this invention in a teeth desensitizing composition application following a teeth whitening/bleaching session. However, understandably, some methods and products are preferred over others for different reasons, including efficacy of desensitization, ease of preparation, ease of application and cost.

A number of toothpastes for sensitive teeth are currently available on the market, such as the Sensodyne line of products, Crest Sensitivity Protection Toothpaste, Butler Toothpaste and Tom's Natural Toothpaste for Sensitive Teeth, which combine desensitizing agents with abrasives and other ingredients in brushable compositions. The desensitizing agents usually work by blocking ionic movement or by remineralizing the teeth which closes off the tubules which traverse the teeth, or in other modes by use of potassium ion-bearing compositions which interfere with or prevent conduction of signals from nerves to the brain. As to the former mode of action, many sensitivity toothpastes use compounds, such as strontium chloride, which are effective to cause minerals in saliva to crystallize and block pores to shield nerves from cold stimuli and other external influences. With respect to the latter mode of action, other desensitizing compositions employ potassium ion containing compounds, such as potassium nitrate and the like, to create a potassium ion flood in an area which is effective to disrupt the transfer of pain signals to the brain. In other instances, oxalate compounds may be rubbed on partially exposed roots of teeth, or high fluoride content mouthwashes may be employed to help reduce tooth sensitivity, or bonding agents may be applied by dentists to close the pores in teeth. While all of such methods are no doubt effective at least to some degree, and certainly plausible for use in the present invention, such brushing and rubbing methods are not the most preferred for use in the inventive method or system following teeth whitening. More specifically, it is preferably desired to maintain a desensitizing composition in constant contact with a user's teeth for a length of time directly following whitening, rather than using a brush to apply a desensitizer, and then to almost immediately brush it or rub it or rinse it away. Further, many people tend to brush with too much pressure which may cause gums to recede and expose dentin which would compound sensitivity problems, and which also may brush away mineral deposits which had formed to block pores, which again defeats any attempt at desensitization.

Therefore, in accordance with the present inventive methods and products, in a preferred embodiment, following the procedure of contacting a user's teeth with the whitening composition for a desired time, a potassium ion-containing composition, preferably in gel form, is next applied to the user's teeth in a manner effective to initiate and provide a prolonged and unbroken contact with teeth and gum areas for a time sufficient to eliminate or at least reduce to a degree teeth and/or gum sensitivity.

The desensitizing compositions suitable for use herein may be in any conventional form, with the employ of any known desensitizing agents or combinations thereof contemplated as well. For example, potassium ion liberating compounds of any sort are contemplated for use, with some non-limiting examples including potassium nitrate, potassium citrate, potassium chloride, potassium bicarbonate, and potassium oxalate, with potassium nitrate preferred for its desensitizing efficacy. The potassium salt is preferably present in the desensitizing composition in range of from about 0.001% by weight to about 20% by weight, and more preferably up to about 5 per cent by weight. Other suitable teeth desensitizing agents which may be used herein or in addition to potassium nitrate include, for example, citric acid, citric acid salts, strontium chloride and the like.

The total amount of desensitizing agents employed, whether or not combined with potassium nitrate, will vary widely, in accordance with such considerations as the desired strength and exposure times. Generally, such a range is preferably from about 0.001 weight % to about 20% by weight, or any range found to be efficacious per unit time, and which may depend upon constituents prior employed and deployed in the whitening composition and those constituents employed in the desensitizing composition. Again, given the knowledge of such compounds in the art, the desired efficacy and appropriate concentration range may be determined without undue effort.

Dental active agents and other adjuvants, excipients and the like may be also be present in the desensitizing composition, which is preferably provided in a gel form. Gels are thought to be advantageous in reaching and maintaining constant and uniform contact with areas sought to be desensitized with potassium ion concentration and/or other desensitizing agent(s). Gels are also advantageous for serving to securely hold in place against teeth and gum areas a dental tray for its uniform application.

As with preferred teeth whitening compositions, suitable gelling agents for use with the desensitizing composition may be any known in the art, with some non-limiting examples including carboxymethylcellulose, carboxypolymethylene, carboxypropyl cellulose, hydroxyethyl cellulose, poloxamer, capanite, natural and synthetic gums, such as, carrageenan, gum karaya, xanthan gum, guan gum, gum arabic, gum tregacanth, veegum, carboxyvinyl and polymers and other examples described hereinabove and mixtures thereof. A preferred gelling agent for use in the desensitizing composition is carboxymethylcellulose, and present in amounts ranging form about 0.001% by weight to about 80% by weight, and more preferably from about 0.10% by weight to about 20% by weight of the desensitizing composition. Other suitable gelling agents may be those of varying water solubility, or of limited water solubility, with some examples including ethyl cellulose and polyox resins.

Thickening agents which may be used, some of which may also act as gelling agents, include both those of the inorganic and organic variety, such as silica compounds, particularly amorphous silica, hydrated silica, carageenan, xantham gum, sodium carboxymethyl cellulose, starch, polyvinylpyrrolidone, hydroxyethlypropyl cellulose, hydroxybutymethyl cellulose, hydroxypropylmethyl cellulose and hydroxyethyl cellulose. Such thickening may be added in any amount to achieve a desired viscosity, or perhaps a particular consistency when employed with a gelling agent.

Additional constituents of the desensitizing composition may be, for example, and without limitation, all manner of dental active agents, excipients and adjuvants, such as water, humectants, surfactants, pH adjusting agents, chelating agents, abrasives, antibacterial, antimicrobial, anti-fungal, anti-cariogenic agents, anti-demineralizing compounds, remineralizing compounds, and flavoring and sweetening agents, such as are discussed above for use with the whitening composition, and which may be present in any amount showing any amount of efficacy for its intended purpose.

As in the teeth whitening compositions, a pH adjusting agent or buffer may be added to the desensitizing composition to ensure its safety for use with oral deployment. The pH adjusting agents or buffers, which can be any material suitable to adjust the pH of the composition, may include, for example the buffer systems based on sodium bicarbonate sodium phosphate, sodium acetate sodium stannate and sodium citrate as well as any other biologically compatible buffer pairs, simple bases such as sodium hydroxide, ammonium hydroxide, potassium hydroxide and triethanolamine, simple acids such as, citric acid, hydrochloric acid, and acetic acid, and combinations thereof. Such pH adjusting material are used in any amount effective to achieve and maintain a desired pH range, such as from about 0.01% to about 10% by weight and which amount will depend upon the other constituents in the composition.

The employ of surfactants in the desensitizing compositions of the invention are particularly useful to provide foaming in some embodiments to ensure that the desensitizing composition reaches cracks and crevices in the teeth and to all of the surface area as desired to be desensitized following a teeth whitening exercise. Any of the several known anionic, cationic, or nonionic surfactants may be used, such as exemplified hereinabove, in any amount efficacious in any way for teeth desensitizing.

Further, as with the whitening composition, some examples of stabilizing, agents which may be employed with the desensitizer include benzoic acid, salicylic acid, butylated hydroxyanisale (BHA), butylatedhydroxytolune (BHT), phosphates and tin salts as well as various parabens including, but not limited to, methyl-, ethyl-propyl and butyl paraben.

Preparation of the desensitizing compositions may be accomplished by any conventional manner. In a preferred embodiment, for example, a suitable desensitizing composition may be prepared by dispersing humectants, as vehicles which may act as moisturizers by attracting moisture to themselves, such as glycerin and polyethylene glycol, with organic thickeners or a gel. A premixed solution of warm water, such as slightly higher temperature than ambient, desensitizing agent, any sweeteners, dyes, pigments, and pH buffer, such as sodium hydroxide and/or sodium bicarbonate and/or sodium carbonate, is next prepared and admixed with the humectant/thickener dispersion to obtain a homogeneous paste or a gel. Optionally, an abrasive, such as a peroxide incompatible abrasive, for example, silica, may be added to the warm water solution, along with flavoring, coloring and surfactant ingredients, and perhaps antimicrobial and/or antibacterial agents and the like as well, prior to gelling. The admixture is preferably processed under a high rate of stirring and under a vacuum to produce a homogeneous gel substantially bereft of air pockets.

The desensitizing composition may be applied to a person's teeth by any conventional or non-conventional manner, such as by rubbing on with a finger, the use of strip applicators and the like, or by a dental tray. However, in similar manner as the teeth whitening compositions, the desensitizing composition is preferably applied by way of a dental tray means, preferably custom manufactured by the person making a teeth impression and the dental tray thereafter professionally manufactured or manufactured in assembly line fashion form such impression.

The dental tray means for use with the teeth whitening and desensitizing in accordance with the invention is preferably a thin-walled, flexible and closely fitting teeth-conforming tray to hold the whitening and desensitizing compositions against a person's teeth for a desired length of time. The trays should conform to the size and shape of a person's teeth, and not exert any undue or excessive mechanical pressure onto a person's teeth or gums.

In conventional manner, teeth whitening systems employ dentist dispensed trays which may be custom fitted teeth whitening or bleaching trays created from teeth impressions produced by the dentist or other licensed or unlicensed dental professional, and which may vary based on local laws and regulations. From such impressions the dentist or other facility creates casts, such as gypsum molds or resin casts, from which the dental tray fabrication will be based. In general, casts made from teeth impressions are covered with a conformable material, such as ethylene vinyl acetate (EVA) or other dental suitable thermoplastic elastomeric comfortable material and the like, which is vacuum formed over the cast and then removed to form trays with personalized teeth mimicking indentations and impressions, or impressed with a user's personal teeth topography. Those trays are then ready for application of teeth whitening gels and/or desensitization gels and other materials and substances. Several days are needed to complete the tray fabrication process, with separate trays fabricated for the upper and lower teeth. Oftentimes additional dental appointments may be necessary to evaluate the fit of the dental trays, and to refine if necessary. Further, at some point, during the dental tray fitting process the dentist will dispense a quantity of teeth whitener, such as in a small syringe or squeeze tube, with instructions as to its use with the trays and with additional follow up appointments to evaluate teeth whitening efforts, and perhaps to dispense desensitizing agents or other dental active compositions when the need arises. Such dental office-based teeth whitening activities can be time consuming and quite costly, but as discussed below are not a prerequisite.

### III. Custom made Dental Trays

In a particularly preferred method and business model of the invention, a person or user may purchase materials from a dental office or retail source, or any source, such as in mail order fashion from an Internet source or other supplier, from which the person manufactures and provides a dental impression of her teeth with or without professional dental advice and/or prescription depending on applicable local, state or federal laws or regulations. As contemplated in the present invention, a user may obtain the inventive teeth whitening/teeth desensitizing materials, including teeth impression making materials, direct from a mail order source, or a user may obtain a prescription for one or more components of the inventive materials from a dental professional licensed in a particular jurisdiction of intended use as required by regulatory requirements, such as the FDA, or equivalent agencies or regulatory bodies. In any event, it is preferred that all products and methods used herein are in full compliance with regulatory agency rules and regulations in whatever jurisdiction and country the inventive products and methods are marketed, sold and used. The self-produced impressions are then forwarded to a facility, for example, in a container or folder with postage pre-paid, for the manufacture of custom fitted trays for use with the teeth whitening and/or teeth desensitizing compositions of the invention. Preferably, the facility will be run on an assembly line model to provide the economical manufacture of casts from teeth impressions using any of the conventional materials available and for the manufacture of custom-fitting dental trays therefrom, again, using any type of acceptable material, such as EVA and the like. However, all other methods for tray fabrication from teeth impressions are also contemplated for use herein. Preferably, the user will receive a set of detailed instructions along with material for producing impressions. Thereafter, the teeth whitening and desensitizing gels are applied with the aid of the custom made trays made from the dental impressions obtained from the customer.

A kit or set of materials for the self-production of teeth impressions typically comprises, along with a set of detailed instructions, an impression—making material for making a negative impression of the upper and lower teeth and optionally a holding means for holding the impression material while the negative impressions are being made and are curing. The holding means may or may not be used, and is not central or critical to the practice of the invention as far as the production of teeth impressions by a user or dental professional is concerned. Any conventional teeth impression material or non-conventional material, or any material effective for creating reproductions of the structure of an individual's teeth and gums, or otherwise personalized teeth topography is contemplated for use herein Such material for making teeth impressions and the like may be used for self-producing the teeth impressions which are the subject of the invention. Impression materials typically used are alginates, which are dental impression materials made from kelp. Alternative impression materials may be comprised of polysulfides, for example organopolysiloxanes and/or polyether products. Alginates are not as preferred as these other products because they distort and alter their shape if positive models are not made within a short period after the negative impression is made. Polymerizable organonpolysiloxanes are generally preferred for use in this invention because of their relatively low cost and their ready availability, and because polyethers are somewhat more difficult to use due to their rigid setting nature and concomitant difficulty in removal from a person's mouth. Polyethers also have an odor and taste that is offensive to some, and are prone to distortion under some conditions such as during sanitizing or disinfecting. Various polymerizable polyorganosiloxane compositions for use in making dental impressions are known, for example, such as those discussed in U.S. Pat. Nos. 6,552,104, 6,550,199 and 6,561,807, all of which may be useful herein. The use of vinylpolysiloxanes is most preferred for use as a casting material. Because these compounds are silicone-based, and are not inherently hydrophilic, many commercially available formulations contain surfactants which lend hydrophilicity to a polymerizable dental impression composition. The surfactant component is also thought to increase wetting characteristics of the polymerizable impression material, with blood and saliva having less of an effect on an impression fabricating activity. Thus, such compositions are preferred for use herein, with particularly preferred compositions employing vinylpolysiloxanes.

### EXAMPLE 1

In a preferred embodiment, an example preparation of a negative dental impression to be used for casting dental trays for use with the whitening and desensitizing compositions of the invention, and/or other bio-active orally deployable compounds and compositions may be conducted as follows:

A set of impression material is provided comprising a vinylpolysiloxane-containing compound in a semi-solid paste consistency, and a catalyst-polymerizing-containing composition which is also provided in a viscous paste composition. Preferably, each composition is of a different color for easy identification purposes. A user combines each of the materials by hand kneading and the like, in a manner such as dough is kneaded, until each of the materials is blended substantially completely into one another and into substantially one color with a total mixing time of less than, for example, about forty-five seconds (the combined material begins to harden in approximately sixty seconds). Once substantially completely combined the resulting composition may be shaped into a rolled form by hand, such as a cylindrical shape, and placed into a provided impression holding tray. The material is then evenly pushed into all portions of the holding tray, preferably in a manner firmly enough such that the material is extruded through one or more small holes in the tray.

As one set of impressions is made per tray, a mold of the upper or lower teeth is first made followed by the other. A holding tray thus filled with impression material is placed, for example, over the center of the upper teeth, and the tray pushed over the teeth with firm pressure, with the impression material surrounding the teeth and gums, such as, for example, one-quarter of an inch, after which the tray is held in place until the impression material sets and hardens to its intended degree. The tray is then removed from the mouth leaving the impression material in the tray. While back teeth may not be included in the impression, it is most preferred that at least the front six teeth and adjacent gum areas are included in the impression. The process is then repeated for the lower teeth, or visa versa depending upon the set of teeth initially molded.

Once manufactured by the user in the above manner, the upper and lower impressions are then forwarded, preferably along with an information/identification card, to a dental tray fabrication facility for custom form fitting dental tray manufacture, after which they are returned to the user for teeth whitening and desensitizing composition application. In yet still another preferred embodiment temporary trays may be provided in the kit to be used until the custom fabricated trays are available for use, which may comprise a teeth conformable impression making composition to be used with the whitening and desensitizing compositions.

In an additional example, a negative dental impression fabrication kit such as above described is provided with whitening and desensitizing compositions in an application means such as a syringe or squeezable tube means comprising each, which may also be applied to provided temporary use trays or the custom fabricated trays for the intended purposes. The kit will also preferably be provided with detailed instructions for its use, and may additionally comprise any other dental active agent, adjuvant or excipient as desired, such as described hereinabove for use with the teeth whitening, hardening and desensitizing compositions.

As can be seen, in accordance with this invention, users may advantageously, with great cost savings, rapidly manufacture their own impressions for a personalized teeth-conforming fit for maximizing uniform and homogeneous contact and application of teeth whitening and desensitizing and remineralizing compounds, and with the assured comfort of custom form fitting devices. A kit means provides all that is needed for a teeth whitening/teeth desensitizing and remineralizing regimen, which completely eliminates the need for costly and unnecessary dental office visits and follow up visits.

In a further preferred embodiment, the dental whitening and desensitizing and remineralizing compositions of the invention are employed to treat a person's teeth in conjunction with conventional dental trays, or the inventive temporary or custom manufactured trays in a manner which exerts negligible mechanical force against a person's teeth or gums. This embodiment is especially afforded by the close-fitting, custom made temporary and mass manufactured custom trays of the invention, which closely conform and/or otherwise register to a user's unique teeth surface profiles inclusive of all crevices and interstitial spaces and the like, and the gingival margin of the user's teeth, with the gelled and/or viscous dental compositions inter-disposed between the tray and teeth, and by virtue of its consistency firmly holding the trays in place for a desired time period. In essence, the inventive molds are retained in place by the gentle action of the gelled compositions, rather than by primarily brute and oftentimes uncomfortable, mechanical force of the trays themselves.

If so desired, the teeth whitening and desensitizing and remineralizing compositions may be manufactured so as to be increasingly tacky and sticky for a more secure attachment of the trays to a person's teeth and gum areas, to ensure homogenous and complete contact. In other words, in some applications it may become desirable that an applied composition be provided as a sticky matrix material to yield a composition having a desired level of stickiness, thus enabling a preferred conformable-fitting dental tray to be more securely held in place over a user's teeth during treatment sessions. Various tackifying agents and components are known to achieve such a result, such as described in International Patent Application No. PCT/US99/26355.

### V. Dental Products and Teeth Whitening Business Methods

As may be further envisioned, the unique advantages afforded by the dental whitening/teeth desensitizing and remineralizing kits and/or system of the present invention provide for equally unique and heretofore unavailable advantages in conducting a dental products and teeth whitening business, especially in a retail mail order model. Now, as afforded by the present invention complete custom made dental tray-endowed whitening systems with a desensitizing and/or remineralizing component and a vast array of other optional dental-active and/or bio-active orally deployable components are made available for public use from any source, including television and Internet retail sales. In this embodiment the present invention provides for a retail mail order teeth whitening and teeth desensitizing business, comprising a teeth whitening service module which provides a context for disseminating materials and instructions for performing a teeth whitening process or method whereby individuals may effectively whiten their teeth (and desensitize and/or remineralize as needed) without any input from a dental office or with prescription from dental professionals, depending, of course, on local regulatory rules and regulations. A patient database model generated from a dental office, or other central location, is also contemplated. If required, such components and materials of the invention may be prescribed by a licensed dental professional, and then made available by mail order or other means to a customer by way of such prescription and inclusive of a database module.

In one example, for instance, the inventive dental tray impression-making materials, whitening compositions, desensitizing compositions, remineralizing compositions and various related products and the like, and assemblies and kits comprising same, may be offered for sale, such as in a saloon, kiosk or other sales point, and orders therefore may be booked/recorded. Such purchase orders are thereafter forwarded to a processing location, such as a central, or a local or regional processing location, for order filling, and preferably through a network system in real time, such as by telephone, facsimile, Internet, or through a PAD, such as a Blackberry device and the like, after which ordered materials are forwarded to the purchaser, depending, of course, upon payment particulars. In other embodiments, depending upon the laws of the jurisdiction in which products and/or services are disseminated and/or sold, orders taken are first forwarded to a licensed dental professional who must first approve and/or otherwise prescribe the inventive dental tray impression-making materials and/or teeth whitening materials and the like, and thereafter the order or prescription may be forwarded to a central processing station for filling and forwarding to a user, or the prescription may be returned to a user directly, or a place of purchase for further processing to the central location for prescription filling, for example, of teeth impression-making material, teeth whitening/teeth desensitizing material and the like. More particularly, a person placing an order may be first asked one or more questions, or be interviewed as to personal dental and/or health conditions, such that a licensed ( or unlicensed) dental professional may ascertain the appropriate selection or prescription of products and/or services, and what is safe and appropriate for a patient. For instance, a potential customer/patient for purposes of ascertaining relevant dental and/or health history may be queried as to the following or related personal information:

(i) Do you have any loose teeth?

(ii) Do you wear braces, and if so for how long?

(iii) Do you have any temporary dental work?

(iv) Do you have any current health problems?

(v) If so, please explain.

(vi) When was the last time you visited a dental office?

(vii) When was the last time you had dental work performed, and what type?

(viii) Do you know if you have any cavities or dental work that is needed?

(ix) Do you now experience teeth sensitivity or pain to cold, heat or various foods?

(x) When did you last have your teeth cleaned?

(xi) Are you allergic to any substances or medications that you know of?

(xii) Have you ever made impressions of you teeth before?

(xiii) Have you undergone teeth whitening or teeth desensitizing before?

(xix) Were you satisfied with the results?

(xx) Have you experienced any problems in the past with teeth whitening?

(xxi) If so, please explain.

The foregoing are only intended to be illustrative of some example questions which may be submitted to a potential customer/patient for prescription purposes, and are not intended to limit this invention in any way. As will be appreciated by those skilled in the art and by dental professionals in general, many variations are possible and are contemplated for use within this invention.

Next, depending upon the information supplied by the potential cutstomer/patient, a prescription may be prepared therefor and forwarded, preferably with the queried information recorded in a database, or the order may receive a negative response, such an electronic “no”, and that teeth whitening materials, such as dental tray fabrication materials, teeth whitening/teeth desensitizing materials and the like may not be appropriate for the potential patient for one or more reasons, all of which may also be entered into a central database. As will also be appreciated by those skilled in the art, database generation and maintenance of customer/patients' dental/medical histories will further facilitate dissemination of future prescriptions for teeth whitening and dental care products in general.

As one may readily observe, the present business method provides the ability to businesses and potential clients alike for the prescription and use of teeth whitening and dental care products, and to provide such products and services to a mass market without the need for expensive and time consuming dental office visits.

As discussed hereinabove, any teeth whitening methods and products, including custom made dental trays from self-made impressions, preferably combined with teeth desensitizing methods and products, may be used in the teeth whitening business model. This method also eliminates, as desired, and described above, preliminary and preparatory activities, such as dental examinations, teeth priming and the placement of gingival isolation materials. Also eliminated, as desired, are follow up activities, such as clean up, consultation and other post teeth whitening processes and activities. The inventive retail mail order teeth whitening service model advantageously includes only those activities, products, procedures and processes related to the actual whitening and optionally including a desensitizing and/or remineralizing of a customer's teeth. Further, unlike a visit to the dentist's office where a patient is allocated a specific time period for treatment, the present inventive business method enables the simultaneous treatment for teeth whitening by many patients, which vastly increases the amounts of products and materials distributed and sold for teeth whitening sessions and/or concomitant teeth desensitizing sessions and the like, all of which vastly increases the profitability of such retail mail order business.

As the inventive teeth whitening business model involves certain discrete steps that allow for direct interaction of a customer with distributors and suppliers of cosmetic dental care products and the like, such interaction may be structured in a way to lead to post-whitening product or service sales ancillary to the actual teeth whitening procedure, such as sales and distribution of remineralizing agents, toothbrushes and toothpaste, breath enhancers, smoking desire inhibitors, alcohol consumption desire inhibitors, vitamins, nutrients and food supplements and the like. Clients may also be encouraged to treat their teeth in a pre-whitening preparatory stage with, for example, a toothbrush or other means which may be packaged in such a way as to clearly associate such dental care product means with brand names and trademarks under which teeth whitening products and services are sold through the business model to generate valuable business good will and source identifying trademark rights.

It is additionally contemplated that the inventive products and methods be marketed and sold in multi-level type home parties, such as Tupperware products and Mary Kay cosmetics, and inclusive of all the embodiments illustrated and identified above.

This invention has been described in conjunction with illustrative examples of some preferred embodiments, but it is not to be construed to be so limited as one skilled in the art will be able to utilize and employ substitutes and equivalents thereof without departing form the scope and bounds of the invention and the spirit thereof.

I claim:

1. A teeth whitening method comprising a person contacting her teeth with a teeth whitening composition for a teeth whitening time period, and then contacting said teeth with a teeth and/or gum desensitizing composition for a desensitizing time period and/or contacting said teeth with said desensitizing composition prior to contact with the whitening composition.

2. The method of claim 1 wherein said teeth whitening composition comprises a teeth whitening active compound selected from the group consisting of hydrogen peroxide, calcium peroxide, glycerol peroxide, benzyl peroxide, carbamide peroxide, sodium percarbonate, potassium percarbonate, and mixtures thereof.

3. The method of claim 2 wherein said teeth whitening active compound is carbamide peroxide present in a teeth whitening amount.

4. The method of claim 1 wherein the desensitizing composition comprises a desensitizing compound selected from the group consisting essentially of potassium nitrate, potassium citrate, potassium chloride, potassium bicarbonate, potassium oxalate and other oxalate compounds, citric acid, citric acid salts, strontium chloride and combinations thereof.

5. The method of claim 4 wherein said desensitizing compound is potassium nitrate, and present in a desensitizing amount.

6. The method of claim 5 wherein the potassium nitrate is present in the desensitizing composition in an amount of from about 0.01% by weight to about 20% by weight of the composition.

7. The method of claim 3 wherein the carbamide peroxide is present in the teeth whitening composition in an amount of from about 0.01% by weight to about 40% by weight of the composition,

8. The method of claim 1 wherein the teeth whitening composition and/or desensitizing composition further comprises a compound selected from the group consisting of water, gelling agents, thickening agents, pH adjusting agents and/or buffers, stabilizers, activators, promoters, catalysts, accelerating agents, flavoring agents, sweetening agents, coloring agents, dyes, chelating agents, abrasive compounds, surfactants, foaming agents and mixtures thereof.

9. The method of claim 8 wherein said gelling and/or thickening agent is selected from the group consisting of polyvinylpyrrolidone, carboxymethylcellulose, carboxypropylcellulose, carboxypolymethylene, hydroxymethylcellulose, sodium carboxymethyl cellulose and other cellulose salts, carrageenan, gum karaya, xanthan gum, guar gum, gum Arabic, gum tragacanth, carboxyvinyl polymers, poloxomer, silica compounds, starches, hydroxyethylpropylcellulose, hydroxybutylmethylcellulose, hydroxypropylmethylcellulose and mixtures thereof.

10. The method of claim 9 wherein the gelling agent is carboxymethylcellulose and is present in an amount ranging from about 0.001% by weight to about 80% by weight of the composition.

11. The method of claim 8 wherein said teeth whitening composition and/or desensitizing composition may additionally comprise remineralizing agents and/or anti-demineralizing agents and mixtures thereof.

12. The method of claim 11 wherein said remineralizing agents are fluoride salts.

13. The method of claim 12 wherein said agents are selected from sodium fluoride and stannous fluoride.

14. The method of claim 8 wherein said teeth whitening composition and/or said desensitizing composition may also comprise one or more anti-cariogenic effective agents.

15. The method of claim 14 wherein said anti-carioogenic agent is xylitol present in an anti-cariogenic amount (sorbitol too).

16. The method of claim 8 wherein said teeth whitening composition and/or said desensitizing composition may additionally comprise one or more compounds selected from antimicrobial, antibacterial, anitbiotic and antifungal effective agents and mixtures thereof, and which is present in any amount which shows efficacy to any degree.

17. The method of claim 16 wherein the compound is selected from the group consisting of chlorohexidine, tetracycline, cetyl pyridinium chloride, benzalkonium chloride, cetyl pyridinium bromide, methyl benzoate, benzoate, penicillins and other classes of antibiotics both of natural and synthetic origin and mixtures thereof.

18. The method of claim 17 wherein said compound is added to the teeth whitening composition and/or desensitizing composition just prior to application of said composition to a user's teeth, or is applied to a user's teeth before, during of after application of said compound.

19. The method of claim 8 wherein said composition(s) may additionally comprise one or more humectants in humectant effective amounts.

20. The method of claim 8 wherein the pH adjusting agent is selected from the group consisting of sodium hydroxide, ammonium hydroxide, potassium hydroxide, sodium stannate, triethanolamine, citric acid, hydrochloric acid, sodium citrate, and combinations thereof, and present in a pH adjusting amount.

21. The method of claim 8 wherein the abrasive compound is selected from the group consisting of dicalcium phosphate dehydrate, anhydrous dicalcium phosphate, calcium pyrophosphate, hydroxy apatite, sodium metaphosphate, potassium metaphosphate, calcium carbonate, sodium bicarbonate, alumina trihydrate, alumina silicate, calcinated alumina and mixtures thereof hydrated silica compounds.

22. The method of claim 21 wherein the abrasive compound in the teeth whitening composition is selected from the group consisting essentially of dicalcium phosphate dehydrate, anhydrous dicalcium phosphate, calcium pyrophosphate and mixtures thereof.

23. The method of claim 8 wherein the chelating compound is selected from the group consisting of sodium acid pyrophosphate, disodium calcium ethylene diamine tetraacetic acid, phosphoric acid, citric acid, sodium citrate, potassium citrate, sodium phosphate, sodium pyrophosphate, potassium stannate, disodium ethylene diamine tetraacetate, disodium ethylene diamine tetraacetic acid and mixtures thereof, and present in a chelating amount.

24. The method claim 8 wherein the surfactant is selected from anionic, cationic and nonionic surfactants and zwitterionic surfactants.

25. The method of claim 8 wherein the coloring agent is a peroxide sensitive dye.

26. A dental product assembly and/or kit comprising a teeth whitening composition and a teeth and/or gum desensitizing composition for application to the teeth before and/or after the application of the teeth whitening composition.

27. The dental product assembly and/or kit of claim 26 wherein the teeth whitening composition comprises a teeth whitening active compound selected from the group consisting of hydrogen peroxide, calcium peroxide, urea peroxide, glycerol peroxide, benzyl peroxide, carbamide peroxide, sodium percarbonate, potassium percarbonate, and mixtures thereof.

28. The dental product assembly and/or kit of claim 27 wherein the teeth whitening active compound is carbamide peroxide.

29. The dental product assembly and/or kit of claim 26 wherein the desensitizing composition comprises a desensitizing compound selected from the group consisting essentially of potassium nitrate, potassium citrate, potassium chloride, potassium bicarbonate, potassium oxalate and other oxalate compounds, citric acid, citric acid salts, strontium chloride, and combinations thereof.

30. The dental product assembly and/or kit of claim 29 wherein said desensitizing compound is potassium nitrate.

31. The dental product assembly and/or kit of claim 30 wherein the potassium nitrate is present in the desensitizing composition in an amount of from about 0.001% by weight to about 50% by weight of the composition.

32. The dental product assembly and/or kit of claim 28 wherein the carbamide peroxide is present in the teeth whitening composition in an amount of from about 0.001% by weight to about 50% by weight of the composition.

33. The dental product assembly and/or kit of claim 26 wherein the teeth whitening composition and/or desensitizing composition additionally comprise a compound selected from the group consisting of water, gelling agents, thickening agents, pH adjusting agents, stabilizers, activators, promoters, catalysts, accelerating agents, flavoring agents, sweetening agents, coloring agents, dyes, chelating compounds, abrasives, surfactants and mixtures thereof.

34. The dental assembly and/or kit of claim 33 wherein said gelling and/or thickening agent is selected from the group consisting of polyvinylpyrrolidone, carboxymethylcellulose, carboxypropylcellulose, carboxypolymethylene, hydroxymethylcellulose, sodiumcarboxymethylcellulose and other cellulose salts, carrageenan, gum karaya, xanthan gum, guar gum, gum Arabic, gum tragacanth, carboxyvinyl polymers, poloxomer, silica compounds, starches, hydroxyethylpropylcellulose, hydroxybutylmethylcellulose, hydroxypropylmethylcellulose and mixtures thereof.

35. The dental product assembly and/or kit of claim 34 wherein the gelling agent is carboxymethylcellulose and is present in an amount of from about 0.001% by weight to about 50% by weight of the composition.

36. The dental product assembly and/or kit of claim 26 wherein said teeth whitening composition and/or desensitizing composition may additionally comprise remineralizing agents and/or anti-demineralizing agents and mixtures thereof.

37. The dental product assembly and/or kit of claim 36 wherein said agents are selected from sodium fluoride and stannous fluoride.

38. The dental product assembly and/or kit of claim 26 wherein said teeth whitening composition and/or said desensitizing composition may additionally comprise one or more anticariogenic effective agents.

39. The dental product assembly and/or kit of claim 38 wherein said anti-cariogenic agent is xylitol and/or sorbitol present in an anti-cariogenic amount.

40. The dental product assembly and/or kit of claim 26 wherein said teeth whitening composition and/or desensitizing composition may additionally comprise one or more compounds selected from the group consisting of antibacterial, antimicrobial, antibiotic and antifungal agents, and mixtures thereof, and present in any amount which shows any efficacy to any degree.

41. The dental product assembly and/or kit of claim 40 wherein the compound is selected from chlorohexidine, tetracycline, cetyl pyridinium chloride, benzalkonium chloride, cetyl pyridinium bromide, methyl benzoate, benzoate, penicillins and other classes of antibiotics of synthetic and/or nonsynthetic origins and mixtures thereof.

42. The dental product assembly and/or kit of claim 40 wherein said compounds are added to the teeth whitening composition and/or desensitizing composition just prior to application of the composition to the teeth, or before, during or after application of the composition to the teeth.

43. The dental product assembly and/or kit of claim 26 wherein said teeth whitening composition and/or desensitizing composition may additionally comprise one or more humectants in a humectant effective amount.

44. The dental product assembly and/or kit of claim 33 wherein said teeth whitening composition and/or desensitizing composition comprises a pH adjusting agent selected from the group consisting of sodium hydroxide, ammonium hydroxide, potassium hydroxide, sodium stannate, triethanolamine, citric acid, hydrochloric acid, sodium citrate, and combinations thereof, and present in a pH adjusting amount.

45. The dental product assembly and/or kit of claim 33 wherein said teeth whitening composition and/or said desensitizing composition may comprise an abrasive compound selected from the group consisting of dicalcium phosphate dehydrate, anhydrous dicalcium phosphate, calcium pyrophosphate, hydroxyapatite, sodium metaphosphate, potassium metaphosphate, calcium carbonate, sodium bicarbonate, alumina trihydrate, alumina silicate, calcinated alumina hydrated silica compounds and mixtures thereof.

46. The dental product assembly and/or kit of claim 45 wherein the abrasive compound present in the teeth whitening composition is selected from the group consisting essentially of dicalcium phosphate dehydrate, anhydrous dicalcium phosphate, calcium pyrophosphate and mixtures thereof.

47. The dental product assembly and/or kit of claim 33 wherein the chelating compound is selected from sodium acid pyrophosphate, disodium calcium ethylene diamine tetraacetic acid, phosphoric acid, citric acid, sodium, citrate, potassium citrate, sodium phosphate, sodium pyrophosphate, potassium pyrophosphate, potassium pyrophosphate, potassium stannate, disodium ethylene diamine tetraacitate, disodium ethylene diamine tetraacetic acid and mixtures thereof.

48. The dental product assembly and/or kit o f claim 33 wherein the surfactant is selected from anionic, cationic and nonionic and zwitterionic compounds, and mixtures thereof.

49. The dental product assembly and/or kit of claim 33 wherein in the teeth whitening composition the coloring agent is a peroxide sensitive dye.

50. A process for the production of a dental product assembly and/or kit of claim 26.

51. A process for the production of a dental product assembly and/or kit of claim 27.

52. A process for the production of a dental product assembly and/or kit of claim 28.

53. A process for the production of a dental product assembly and/or kit of claim 29.

54. A process for the production of a dental product assembly and/or kit of claim 30.

55. A process for the production of a dental product assembly and/or kit of claim 31.

56. A process for the production of a dental product assembly and/or kit of claim 32.

57. A process for the production of a dental product assembly and/or kit of claim 33.

58. A process for the production of a dental product assembly and/or kit of claim 34.

59. A process for the production of a dental product assembly and/or kit of claim 35.

60. A process for the production of a dental product assembly and/or kit of claim 36.

61. A process for the production of a dental product assembly and/or kit of claim 37.

62. A process for the production of a dental product assembly and/or kit of claim 38.

63. A process for the production of a dental product assembly and/or kit of claim 39.

64. A process for the production of a dental product assembly and/or kit of claim 40.

65. A process for the production of a dental product assembly and/or kit of claim 41.

66. A process for the production of a dental product assembly and/or kit of claim 42.

67. A process for the production of a dental product assembly and/or kit of claim 43.

68. A process for the production of a dental product assembly and/or kit of claim 44.

69. A process for the production of a dental product assembly and/or kit of claim 45.

70. A process for the production of a dental product assembly and/or kit of claim 46.

71. A process for the production of a dental product assembly and/or kit of claim 47.

72. A process for the production of a dental product assembly and/or kit of claim 48.

73. A process for the production of a dental product assembly and/or kit of claim 49.

74. A method for treating a person in need thereof comprising the application of a dental product assembly and/or kit of any of claims 26, 27, 28, 29, 30, 31, 32, 33, 34, 35, 36, 37, 38, 39, 40, 41, 42, 43, 44, 45, 46, 47, 48 and 49.

75. The dental product assembly and/or kit of any of claims 26, 27, 28, 29, 30, 31, 32, 33, 34, 35, 36, 37, 38, 39, 40, 41, 42, 43, 44, 45, 46, 47, 48 and 49 further comprising means for a person's production of dental impressions which are effective for the transformation thereof into custom fitted dental trays for application of the teeth whitening composition and/or desensitizing composition.

76. A process for the production of the dental product assembly and/or kit of claim 75.

77. A method for treating a person in need thereof with the dental product assembly of claim 75.

78. The dental product assembly and/or kit of claim 75 further comprising a temporary means for a person's self application of said teeth whitening composition and/or desensitizing composition.

79. A process for the production of the dental product assembly and/or kit of claim 78.

80. A method for treating a person in need thereof with the dental product assembly of claim 78.

81. The dental product assembly of claim 75 wherein said means for a person's production of said dental impression comprises polysulfides, as for example, polymerizable vinylpolysiloxane compound and a catalyst-containing compound effective for polymerizing said vinylpolysiloxane compound.

82. A method for treating a person in need thereof with the dental product assembly of claim 81.

83. A method of conducting one or more business functions selected from designing, manufacturing, marketing, using, selling, licensing, leasing, generating business good will, generating trademark rights and forming and operating a business entity comprising the use of a teeth whitening method which comprises a person contacting her teeth with a teeth whitening composition for a teeth whitening time period, then contacting said teeth with a teeth and/or gum desensitizing composition for a desensitizing time period and/or contacting said teeth with said desensitizing composition prior to contact with the whitening composition.

84. The method of claim 83 wherein said teeth whitening composition comprises a teeth whitening active compound selected from the group consisting of hydrogen peroxide, calcium peroxide, glycerol peroxide, benzyl peroxide, carbamide peroxide, sodium percarbonate, potassium percarbonate, and mixtures thereof.

85. The method of claim 84 wherein said teeth whitening active compound is carbamide peroxide present in a teeth whitening amount.

86. The method of claim 83 wherein the desensitizing composition comprises a desensitizing compound selected from the group consisting essentially of potassium nitrate, potassium citrate, potassium chloride, potassium bicarbonate, potassium oxalate and other oxalate compounds, citric acid, citric acid salts, strontium chloride and combinations thereof.

87. The method of claim 83 wherein said desensitizing compound is potassium nitrate, and present in a desensitizing amount.

88. The method of claim 87 wherein the potassium nitrate is present in the desensitizing composition in an amount of from about 0.001% by weight to about 50% by weight of the composition.

89. The method of claim 84 wherein the carbamide peroxide is present in the teeth whitening composition in an amount of from about 0.001% by weight to about 50% by weight of the composition,

90. The method of claim 83 wherein the teeth whitening composition and or desensitizing composition further comprises a compound selected from the group consisting of water, gelling agents, thickening agents, pH adjusting agents, stabilizers, activators, promoters, catalysts, accelerating agents, flavoring agents, sweetening agents, coloring agents, dyes, chelating agents, abrasive compounds, surfactants and mixtures thereof.

91. The method of claim 90 wherein said gelling and/or thickening agent is selected from the group consisting of polyvinylpyrrolidone, carboxymethylcellulose, carboxypropylcellulose, carboxypolymethylene, hydroxymethylcellulose, sodium carboxymethyl cellulose and other cellulose salts, carrageenan, gum karaya, xanthan gum, guar gum, gum Arabic, gum tragacanth, carboxyvinyl polymers, poloxomer, silica compounds, starches, hydroxyethylpropylcellulose, hydroxybutylmethylcellulose, hydroxypropylmethylcellulose and mixtures thereof.

92. The method of claim 91 wherein the gelling agent is carboxymethylcellulose and present in an amount ranging from about 0.001% by weight to about 50% by weight of the composition.

93. The method of claim 90 wherein said teeth whitening composition and/or desensitizing composition may additionally comprise remineralizing agents and/or anti-demineralizing agents and mixtures thereof.

94. The method of claim 93 wherein said remineralizing agents are fluoride salts.

95. The method of claim 94 wherein said agents are selected from sodium fluoride and stannous fluoride.

96. The method of claim 90 wherein said teeth whitening composition and/or said desensitizing composition may also comprise one or more anti-cariogenic effective agents.

97. The method of claim 96 wherein said anti-carioogenic agent is xylitol present in an anti-cariogenic amount.

98. The method of claim 90 wherein said teeth whitening composition and/or said desensitizing composition may additionally comprise one or more compounds selected from antimicrobial, antibacterial and antifungal effective agents and mixtures thereof, and which is present in any amount which shows efficacy to any degree.

99. The method of claim 98 wherein the compound is selected from the group consisting of chlorohexidine, tetracycline, cetyl pyridinium chloride, benzalkonium chloride, cetyl pyridinium bromide, methyl benzoate, benzoate, and mixtures thereof.

100. The method of claim 99 wherein said compound is added to the teeth whitening composition and/or desensitizing composition just prior to application of said composition to a user's teeth, or is applied to a user's teeth before, during of after application of said compound.

101. The method of claim 9 wherein said composition(s) may additionally comprise one or more humectants in humectant effective amounts.

102. The method of claim 90 wherein the pH adjusting agent is selected from the group consisting of sodium hydroxide, ammonium hydroxide, potassium hydroxide, sodium stannate, triethanolamine, citric acid, hydrochloric acid, sodium citrate, and combinations thereof, and present in a pH adjusting amount.

103. The method of claim 90 wherein the abrasive compound is selected from the group consisting of dicalcium phosphate dehydrate, anhydrous dicalcium phosphate, calcium pyrophosphate, hydroxy apatite, sodium metaphosphate, potassium metaphosphate, calcium carbonate, sodium bicarbonate, alumina trihydrate, alumina silicate, calcinated alumina and mixtures thereof.

104. The method of claim 103 wherein the abrasive compound in the teeth whitening composition is selected from the group consisting essentially of dicalcium phosphate dehydrate, anhydrous dicalcium phosphate, calcium pyrophosphate and mixtures thereof.

105. The method of claim 90 wherein the chelating compound is selected from the group consisting of sodium acid pyrophosphate, disodium calcium ethylene diamine tetraacetic acid, phosphoric acid, citric acid, sodium citrate, potassium citrate, sodium phosphate, sodium pyrophosphate, potassium stannate, disodium ethylene diamine tetraacetate, disodium ethylene diamine tetraacetic acid and mixtures thereof, and present in a chelating amount.

106. The method claim 90 wherein the surfactant is selected from anionic, cationic and nonionic surfactants.

107. The method of claim 90 wherein the coloring agent is a peroxide sensitive dye.

108. A method of conducting one or more business functions selected from designing, manufacturing, using, marketing, selling, licensing, leasing, generating business good will generating trademark rights and forming and operating a business entity comprising the use of a dental product assembly and/or kit comprising a teeth whitening composition and a teeth and/or gum desensitizing composition for application to the teeth before and/or after the application of the teeth whitening composition.

109. The method of claim 108 wherein the teeth whitening in the dental product assembly and/or kit of claim 26 wherein the teeth whitening composition comprises a teeth whitening active compound selected from the group consisting of hydrogen peroxide, calcium peroxide, urea peroxide, glycerol peroxide, benzyl peroxide, carbamide peroxide, sodium percarbonate, potassium percarbonate, and mixtures thereof.

110. The dental product assembly and/or kit of claim 27 wherein the teeth whitening active compound is carbamide peroxide.

111. The method of claim 108 wherein in said dental product kit, the desensitizing composition comprises a desensitizing compound selected from the group consisting essentially of potassium nitrate, potassium citrate, potassium chloride, potassium bicarbonate, potassium oxalate and other oxalate compounds, citric acid, citric acid salts, strontium chloride, and combinations thereof.

112. The method of claim 111 wherein in said dental product kit said desensitizing compound is potassium nitrate.

113. The method of claim 112 wherein in said dental product kit the potassium nitrate is present in the desensitizing composition in an amount of from about 0.001% by weight to about 50% by weight of the composition.

114. The method of claim 110 wherein in said dental product kit the carbamide peroxide is present in the teeth whitening composition in an amount of from about 0.001% by weight to about 50% by weight of the composition.

115. The method of claim 108 wherein in said dental product kit the teeth whitening composition and/or desensitizing composition additionally comprises a compound selected from the group consisting of water, gelling agents, thickening agents, pH adjusting agents, stabilizers, activators, promoters, catalysts, accelerating agents, flavoring agents, sweetening agents, coloring agents, dyes, chelating compounds, abrasives, surfactants and mixtures thereof.

116. The method of claim 115 wherein in said dental product kit said gelling and/or thickening agent is selected from the group consisting of polyvinylpyrrolidone, carboxymethylcellulose, carboxypropylcellulose, carboxypolymethylene, hydroxymethylcellulose, sodiumcarboxymethylcellulose and other cellulose salts, carrageenan, gum karaya, xanthan gum, guar gum, gum Arabic, gum tragacanth, carboxyvinyl polymers, poloxomer, silica compounds, starches, hydroxyethylpropylcellulose, hydroxybutylmethylcellulose, hydroxypropylmethylcellulose and mixtures thereof.

117. The method of claim 116 wherein in said dental product kit the gelling agent is carboxymethylcellulose and is present in an amount of from about 0.001% by weight to about 50% by weight of the composition.

118. The method of claim 111 wherein in said dental product kit said teeth whitening composition and/or desensitizing composition additionally comprises remineralizing agents and/or anti-demineralizing agents and mixtures thereof.

119. The method of claim 118 wherein in said dental product kit said agents are selected from sodium fluoride and stannous fluoride.

120. The method of claim 111 wherein in said dental product kit said teeth whitening composition and/or said desensitizing composition additionally comprises one or more anticariogenic effective agents.

121. The method of claim 120 wherein in said dental product kit said anti-cariogenic agent is xylitol present in an anti-cariogenic amount.

122. The method of claim 111 wherein in said dental product kit said teeth whitening composition and/or desensitizing composition additionally comprises one or more compounds selected from the group consisting of antibacterial, antimicrobial and antifungal agents, and mixtures thereof, and present in any amount which shows any efficacy to any degree.

123. The method of claim 122 wherein in said dental product kit the compound is selected from chlorohexidine, tetracycline, cetyl pyridinium chloride, benzalkonium chloride, cetyl pyridinium bromide, methyl benzoate, benzoate, and mixtures thereof.

124. The method of claim 122 wherein in said dental product kit said compounds are added to the teeth whitening composition and/or desensitizing composition just prior to application of the composition to the teeth, during or after application of the composition to the teeth.

125. The method of claim 111 wherein in said dental product kit said teeth whitening composition and/or desensitizing composition additionally comprises one or more humectants in a humectant effective amount.

126. The method of claim 115 wherein in said dental product kit said teeth whitening composition and/or desensitizing composition comprises a pH adjusting agent selected from the group consisting of sodium hydroxide, ammonium hydroxide, potassium hydroxide, sodium stannate, triethanolamine, citric acid, hydrochloric acid, sodium citrate, and combinations thereof, and present in a pH adjusting amount.

127. The method of claim 115 wherein in said dental product kit said teeth whitening composition and/or said desensitizing composition comprises an abrasive compound selected from the group consisting of dicalcium phosphate dehydrate, anhydrous dicalcium phosphate, calcium pyrophosphate, hydroxyapatite, sodium metaphosphate, potassium metaphosphate, calcium carbonate, sodium bicarbonate, alumina trihydrate, alumina silicate, calcinated alumina and mixtures thereof.

128. The method of claim 127 wherein in said dental product kit, the abrasive compound present in the teeth whitening composition is selected from the group consisting essentially of dicalcium phosphate dehydrate, anhydrous dicalcium phosphate, calcium pyrophosphate and mixtures thereof.

129. The method of claim 115 wherein in said dental product kit, the chelating compound is selected from sodium acid pyrophosphate, disodium calcium ethylene diamine tetraacetic acid, phosphoric acid, citric acid, sodium, citrate, potassium citrate, sodium phosphate, sodium pyrophosphate, potassium pyrophosphate, potassium pyrophosphate, potassium stannate, disodium ethylene diamine tetraacitate, disodium ethylene diamine tetraacetic acid and mixtures thereof.

130. The method of claim 115 wherein in said dental product kit, the surfactant is selected from anionic, cationic and nonionic compounds, and mixtures thereof.

131. The method of claim 115 wherein in said dental product kit in the teeth whitening composition the coloring agent is a peroxide sensitive dye.

132. A method for conducting one or more business functions selected from designing, manufacturing, using, marketing, selling, licensing, leasing, generating business, goodwill, generating trademark rights, and forming and operating a business entity comprising the use of a process for the production of a dental product assembly and/or kit of claim 27.

133. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 27.

134. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 28.

135. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 29.

136. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 30.

137. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 31.

138. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 32.

139. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 33.

140. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 34.

141. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 35.

142. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 36.

143. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 37.

144. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 38.

145. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 39.

146. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 40.

147. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 41.

148. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 42.

149. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 43.

150. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 44.

151. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 45.

152. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 46.

153. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 47.

154. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 48.

155. A method for conducting one or more business functions selected from designing, manufacturing, using, marketing, selling, licensing, leasing, generating business good will generating trademark rights, and forming and operating a business entity comprising the use of. A process for the production of a dental product assembly and/or kit of claim 49.

156. A method for conducting one or more business functions comprising the use of a method for treating a person in need thereof comprising the application of a dental product assembly and/or kit of any of claims 26, 27, 28, 29, 30, 31, 32, 33, 34, 35, 36, 37, 38, 39, 40, 41, 42, 43, 44, 45, 46, 47, 48 and 49.

157. A method for conducting one or more business functions comprising the use of the dental product assembly and/or kit of any of claims 26, 27, 28, 29, 30, 31, 32, 33, 34, 35, 36, 37, 38, 39, 40, 41, 42, 43, 44, 45, 46, 47, 48 and 49 further comprising means for a person's production of dental impressions which are effective for the transformation thereof into custom fitted dental trays for application of the teeth whitening composition and/or desensitizing composition.

158. A method for conducting one or more business functions comprising the use of a process for the production of the dental product assembly and/or kit of claim 157.

159. A method for conducting one or more business functions comprising the use of a method for treating a person in need thereof with the dental product assembly of claim 157.

160. A method for conducting one or more business functions comprising the use of the dental product assembly and/or kit of claim 157 further comprising a temporary means for a person's self application of said teeth whitening composition and/or desensitizing composition.

161. A method for conducting one or more business functions comprising the use of a process for the production of the dental product assembly and/or kit of claim 78.

162. A method for conducting one or more business functions comprising the use of a method for treating a person in need thereof with the dental product assembly of claim 78.

163. A method for conducting one or more business functions comprising the use of the dental product assembly of claim 75 wherein said means for a person's production of said dental impression comprises a polymerizable vinylpolysiloxane compound and a catalyst-containing compound effective for polymerizing said vinylpolysiloxane compound.

164. A method for conducting one or more business functions comprising the use of a method for treating a person in need thereof with the dental product assembly of claim 81.
